(19) United States
(12) Patent Application Publication
(10) Pub. No.: US 2002/0032325 A1
(43) Pub. Date: Mar. 14, 2002

(54) 4-amino-3-mercapto-1,2,4-triazoles

(76) Inventors: Ned D. Heindel, Easton, PA (US); Jeffrey D. Laskin, Piscataway, NJ (US); Diane E. Heck, Rumson, NJ (US); Robert D. Rapp, Reading, PA (US); Christophe Guillon, Bethlehem, PA (US); Angela M. Mendel, Deptford, NJ (US)

Correspondence Address: Competitive Technologies, Inc., 1960 Bronson Road, Fairfield, CT, 06430, US

(21) Appl. No.: 09/790,330

(22) Filed: Feb. 21, 2001

(30) Foreign Application Priority Data
Aug. 21, 1999 (US) PCT/US99/19146

(51) Int. Cl. C07D 285/14; C07D 285/36

(52) U.S. Cl. 540/545; 548/263.2; 544/10; 548/136

(57) ABSTRACT

Synthesis, nitric oxide synthase inhibition, and antiproliferative activity of eight structural variants of functionalized 4-amino-3-mercapto-1,2,4-triazoles, specifically

4-amino-3-mercapto-triazoles;

3-R-8-aryl-1,2,4-triazolo[3,4-b]-1,3,4-thiadiazepines;

3-R-8-aryl-5,6-dihydro-1,2,4-triazolo[4,5-b]-1,3,4-thiadiazepines;

4-amino-3-(R′-mercaptyl)-5-R-(4H)-1.2.4-triazoles;

4-(R′-imino)-3-mercapto-5-(R)-4H-1.2.4-triazoles;

3-(R)-6-(R′)-1,2,4-triazolo-[3,4-b]-1,3,4-thiadiazines;

1,2,4-triazolo[3,4-b]-1,3,4-thiadiazoles; and

4-(R′-imino)-3-alkylthio-5-R-1,2,4-triazoles

are described. These variants of 4-amino-3-mercapto-1,2,4-triazoles are effective as inhibitors of nitric oxide synthase and malignant cellular proliferation.

From a clinical perspective, it is widely believed that the in vivo pharmacological manipulation of nitric oxide (NO) production will be of considerable therapeutic value.

The list of nitric oxide synthase (NOS) mediated diseases becomes longer every year. The broad classes of dysfunctions involving NOS now includes many gastrointestinal motility problems, inflammatory states, and neurodegenerative disorders. A partial listing of specific medical circumstances which appear to be associated with NOS involvement include sunburn, rheumatoid arthritis, ulcerative colitis, Crohn's disease lupus, septic and toxic shock, asthma, hypertension, myocarditis, diabetes, and many autoimmune and respiratory disorders [see Annual Reports in Medicinal Chemistry, J. A. Bristol editor, 31:221 (1996)].

Recently it has become known that the various isoforms of NOS utilize the arginine to citrulline deamination as the route to NO, and many therapeutic drugs have been designed to target that pathway [see Medicinal Research Reviews 14:23 (1994)]. A wide variety of N-gamma-substituted arginines identified as inhibitors of NOS bearing such pendant gamma residues as nitro, amino, and even alkyl, and with the observation that some heterocyclic triazole systems appear to mimic the guanidino portion of arginine [see Biochemical and Biophysical Research Communications 183(1):150 (1992)]. Furthermore, while 1,2,4-triazoles do have an abundant patent literature base as useful agriculturals and even as human therapeutics [see, for example, U.S. Pat. Nos. 5,770,616; 5,756,522; 5,629,322; 5,602,153; 5,470,984; 5,451,591; and 5,382,674], the specific prior art on the 4-amino-1,2,4-triazoles fails to indicate that they possessed inhibitory activity against NOS [see Biochemical and Biophysical Research Communications 183(1):150 (1992)].

In view of this background, we disclose the use of planar, fused-ring bio-isosteric models of arginine as new candidate classes of NOS inhibitors. In the research leading to the present invention, we found significant NOS-inhibitory activity in that 4-amino-1,2,4-triazole family bearing a pendant 3-mercapto moiety. Furthermore, active NOS inhibitors were also found and in several of the N- or S-functionalized derivatives of these 4-amino-3-mercapto-(4H)-1,2,4-triazoles as well as in fused-ring heterocyclic derivatives.

We believe that these heterocyclic candidate therapeutics are functioning as cyclic biological isosteres of the N-aminoguanidines which have previously shown to

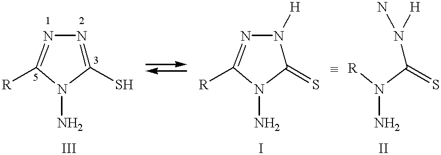

possess NOS inhibition [see Annual Reports in Medicinal Chemistry, J. A. Bristol editor, 31:221 (1996)].

Accordingly, the present invention lies in the synthesis, structure, and utility of eight novel structural variants: 4-amino-3-mercapto-triazoles; 3-R-8-aryl-1,2,4-triazolo[3,4-b]-1,3,4-thiadiazapines; 3-R-8-aryl-5,6-dihydro-1,2,4-triazolo[4,5-b]-1,3,4-thiadiazapines; 4-amino-3-(R′-mercaptyl)-5-R-(4H)-1.2.4-triazoles; 4-(R′-imino)-3-mercapto-5-(R)-4H-1.2.4-triazoles; 3-(R)-6-(R′)-1,2,4-triazolo-[3,4-b]-1,3,4-thiadiazines; 1,2,4-triazolo[3,4-b]-1,3,4-thiadazoles; and 4-(R′-imino)-3-alkylthio-5-R-1,2,4-triazoles as inhibitors of NOS and malignant cellular proliferation. This fundamental molecular construct operates as a heterocyclic mimic of the open-chain N-aminoarginines (or N-aminoguanidines) previously established as NOS inhibitors. In addition, novel processes are described to obtain diverse members of these 4-amino-3-mercapto-1,2,4-triazoles. A convenient method, using PAM 212 keratinocytes, which correlates highly with the detection and quantification of relative NOS inhibition potential in a series of candidate drugs, is also described as a bioassay for determining cellular growth inhibition, and for predicting pharmaceutical activity when these variants are brought into in vivo contact with malignant cells.

In each of the structures depicted herein, R and R′, which may be the same or different, are alkyl, aryl, hydrogen, fluoroalkyl, or heterocyclic moieties. By alkyl is meant any monovalent radical having the structure CₙH₂ₙ₊₁—, especially lower alkyl radicals of between 1 and 6 carbons in length; by aryl is meant any organic radical derived from an aromatic hydrocarbon by the removal of one atom, for example phenyl or substituted phenyl radicals; by haloalkyl is meant a alkyl radical, especially a lower alkyl radical which carries a halide moiety as for example a fluoroalkyl, bromoalkyl, or chloroalkyl; and by heterocyclic is meant a cyclic ring structure, especially a heterocyclic structure having from 5 to 8 atoms in the ring. Especially, among the radicals included in the broad definition of these moieties are hydrogen, bromine, chlorine, methyl, cyclohexyl, phenyl, 2-thienyl, 2-furyl, 3-pyridyl, 2-phenylethyl, trifluoromethyl, C₆H₅—, p—F—C₆H₄—, 4-F—C₆H₄—, 2-Br—C₆H₄—, o-hydroxyphenyl, 2,3-dihydroxyphenyl, β-Me-butyrate, β-phenyl-butyrate, β-phenylpropionate methyl ester, 4-hydroxy-2-butyl, 4-chloro-2-butyl, Ph-CH₂CH₂—, cinnamaldehyde, —CH₂C H₂COOMe, —CH(CH₃)CH₂CH₂Cl, —CH(CH₃)CH₂CO₂(C₆H₅), —CH═CH-Ph, —CH═CH-2-methoxyphenyl, —CH═CH-2-nitrophenyl, —CH═CH-(o-methoxyphenyl), α-bromocinnamaldehyde, —CH═CH-(o-nitrophenyl), α-chlorocinnamaldehyde, and α-methylcinnamaldehyde. More particularly, R may be selected from the group of methyl, cyclohexyl, phenyl, 2-thienyl, 2-furyl, 3-pyridyl, 2-phenylethyl, C₆H₅—, p—F—C₆H₄—, 4-F—C₆H₄—, o-hydroxyphenyl, Ph—CH₂CH₂—, CH═CH—Ph, -nitrophenyl, and 2-Br—C₆H₄—; and R′ may be selected from the group of hydrogen, bromine, chlorine, phenyl, 2-phenylethyl, C₆H₅—, p—F—C₆H₄—, 4-F—C₆H₄—, 2-Br—C₆H₄—, o-hydroxyphenyl, Ph-CH₂CH₂—, cinnamaldehyde, —CH₂CH₂COOMe, —CH(CH₃)CH₂CH₂Cl, —CH(CH₃)CH₂CO₂(C₆H₅), —CH═CH—Ph, —CH═CH-2-methoxyphenyl, —CH═CH-2-nitrophenyl, —CH═CH-(o-methoxyphenyl), α-bromocinnamaldehyde, —CH═CH-(o-nitrophenyl), α-chlorocinnamaldehyde, and α-methylcinnamaldehyde. In addition to these specified radicals, others may appear within the following examples.

More specifically, the present invention describes a syntheses generating unique N- and S-functionalized derivatives of these 4-amino-3-mercapto-4H-1,2,4-triazoles, viz. the 4-amino-3-R′-mercaptyl-5-R-1,2,4-triazoles (general formula VI, in which R′ is not H):

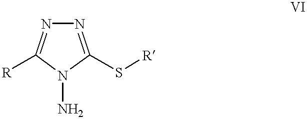

the 4-(R′-imino)-3-mercapto-5-R-1,2,4-triazoles (general formula VII):

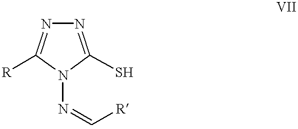

and the 4-(R′-imino)-3-alkylthio-5-R-1,2,4-triazoles (general formula X):

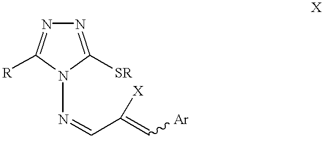

Also described in the present invention is the syntheses of four classes of fused-ring triazole heterocyclics. First, by two synthetic routes, there is described members of a five-seven fused ring system, specifically 3-R-8-aryl-1,2,4-triazolo[3,4-b]-1,3,4-thiadiazepines (general formula IV):

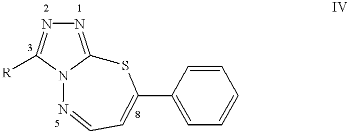

and their reduced 5,6-dihydro counter-parts (general formula V):

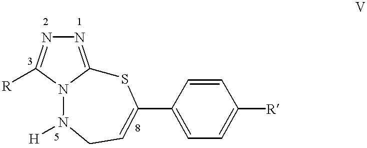

Second, the present invention describes a five-six fused ring system, specifically 3-(R)-6-(R′)-1,2,4-triazolo-[3,4-b]-1,34-thiadiazine (general formula VIII):

In this one-step (indirect fashion, which we refer to as ‘method 2’) the following compounds belonging to the class defined by general formula IV were obtained:

| Compound | R | R′ | yield (%) | mp (° C.) |
|---|---|---|---|---|
| IVa | C₆H₅— | H | 49 | |
| IVb | p-F—C₆H₄— | H | 79 | |
| IVc | 2-thienyl | H | 46 | |
| IVd | methyl | H | 44 | 212-215 |
| IVe | o-hydroxy- | H | 57 | 218-220 |
| | phenyl | | | |

As seen in this second table, the conversions using the single-step method 2 according to the present invention gave an efficiency of conversion (yields for compounds IVa-c) which is in significant excess to that found by prior method 1. Thus, by comparable application of method 2 with any appropriate mercaptoaminotriazole of general formula II and with any appropriate arylpropargylacetal, these triazoles of general formula IV according to the present invention can be obtained in an approximate 40 to 80 percent conversion.

### EXAMPLE3

### Preparation of 3-R-8-aryl-5,6-dihydro-1,2,3-triazolo[4,5-b]-1,3,4-thiadiazepines

### Thiadiazepines of General Formula V

Anhydrous methanol (100 ml) was saturated with dry HCL gas at 0° C. and 1.00 g (3.00 moles) of a thiadiazepine of general formula IV were added with magnetic stirring. To this yellow solution were added 3.80 g (0.10 moles) of solid sodium borate in small portions over a 10 minute period. The mixture was maintained at 0° C. for three hours, heated at reflux for 0.5 hours, and then allowed to cool to room temperature. Evaporation to dryness in vacuo was followed by the addition of 150 ml of cold water, filtration in vacuo, and trituration of the solid product with 200 ml of cold 5% aqueous NaOH, 2×50 ml portions of cold water. Recrystallization from anhydrous methanol gave the titled products in 50-75% conversions. All of these substances, i.e., the 3-R-8-aryl-5,6-dihydro-1,2,3-triazolo[4,5-b]-1,3,4-thiadiazepines prepared according to this example,

Hydrolysis in dilute aqueous sulfuric acid liberated the free arylpropargyl aldehydes in yields of 30-45% starting from the arylacetylene. Aldehydes were extracted into an ether layer, the ether washed with 10% aqueous sodium bicarbonate and then with saturated aqueous sodium chloride, dried over magnesium sulfate, and evaporated in vacuo to the aldehyde. This is generally depicted in the following reaction scheme:

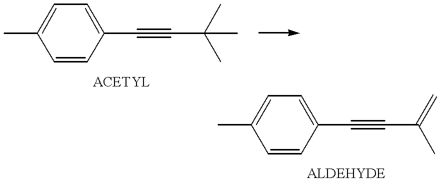

In this fashion, phenylpropargyl aldehyde was obtained in 45% yield; p-chlorophenylpropargyl aldehyde in 32% yield; and p-bromopropargyl aldehyde in 43% yield. The bromo and chloro compounds were not distilled but were used directly as semi-solid oils displaying a characteristic singlet aldehyde ¹H NMR signal at 9.39±0.08 ppm (in CDCl₃) and a strong C═O stretch at 1660±7 cm⁻¹ in the IR spectrum.

b) Condensation of the 4-amino-3-mercapto-1,2,4-triazole:

A solution of 0.10 moles of a requisite 5-R-4-amino-3-mercapto-4H-1.2.4-triazole (general formula III) in 150-200 ml of anhydrous ethanol was degassed by bubbling with nitrogen for 10 minutes. An equimolar quantity of the arylpropargyl aldehyde obtained above in 50 ml of ethanol was added in equal portions over a 30 minute period with magnetic stirring under a constant nitrogen blanket. Agitation at ambient temperature was continued for 24 hours during which time turbidity and some solid precipitation occurred. The mixture was heated to reflux for one hour, evaporated in vacuo to about 100 ml total volume, chilled in an ice/salt bath, and the resulting solid removed by filtration. The solid was titrated with 150 ml of 10% aqueous potassium hydroxide to remove unreacted starting material, and the 3-R-8-aryl-1,2,4-triazolo[3,4-b]-1,3,4-thiadiazepines (according to general formula IV) was subsequently recrystallized from ethanol to analytical purity. All products could be recognized by their characteristic vicinal C-H resonances on the thiadiazepine ring at 6.80±0.10 and 8.27±0.10 ppm (J=4 Hz). In this two-step (indirect fashion, which we refer to as ‘method 1’, were obtained the following compounds of general formula IV:

displayed (in their proton NMR) the characteristic methylene multiplet as an apparent double-doublet at 4.10±0.10 ppm. The reduction of any appropriate member of the class defined by general formula IV generates the corresponding dihydro member of the class defined by general formula V.

Utilizing the procedure outlined above, the following members of the class defined by general formula V were obtained:

| Compound | R | R′ | yield (%) | mp (° C) |
|---|---|---|---|---|
| Vb | phenyl | Br | 69 | >240 (decomp) |
| Vc | 2-thienyl | H | 51 | >230 (decomp) |
| Va* | phenyl | H | 72 | 222-223 |

*Additional findings for compound Va include IR (KBr) 3190, 3020, 1490, 1465, 1445, 1295, 1010, 850, 805, 755, and 690 cm⁻¹; H NMR (DMSO-d₆) 4.06(m, 2H, —CH₂—), 6.08(t, 1H, C═CH, J=3Hz), and 6.00-7.20(m, 11H, ArH + NH).

### EXAMPLE 4

### Preparation of 4-amino-3-(R′-mercaptyl)-5-(R)-(4H)-1,2,4-triazole

### Triazoles of General Formula VI

To generate members of the class defined by general formula VI, a base-catalyzed Michael addition of the tautomeric mercapto moiety in any member of the class identified by general formula III may be effected onto an activated double bond in crotonates, acrylates, cinnamates, and other conjugated alkenyl esters. For example, 4-amino-3-mercaptyl-(beta-methyl-butyrate)-5-(2-thienyl)-(4H)-1,2,4-triazole (compound VIa, below) was prepared by first dissolving 500 mg (2.52 mmol) of 4-amino-3-mercapto-5-(2-thienyl)-(4H)-1,2,4-triazole in 4 ml of dioxane. Subsequently, 10 drops of piperidine were added and the reaction mixture was stirred for 20 minutes at room temperature. Methyl crotonate, 0.535 ml (5.04 mmol), was added and the reaction mixture was heated at reflux for four days. The medium was cooled to room temperature and the excess solvent was removed under reduced pressure. The product was purified by silica gel column chromatography, mobile phase CH₂Cl₂, to give 488 mg of a pale yellow solid with IR (nujol)

It is still another aspect of the present invention to report on the inhibition of nitric oxide synthase for members of all heterocyclic classes of triazoles described herein (i.e., triazoles having structures depicted as general formulae III to X).

It is still another aspect of the present invention to report on the inhibition of cellular growth for members of compounds depicted as general formulae VIII and IX.

A more complete understanding of these and other aspects, terms, and scope of the present invention may be obtained in reference to the following detailed description, figures and examples, all of which are illustrative of the present invention and are not to be taken as limiting the scope and breadth of the present invention in any manner.

In respect to the figures:

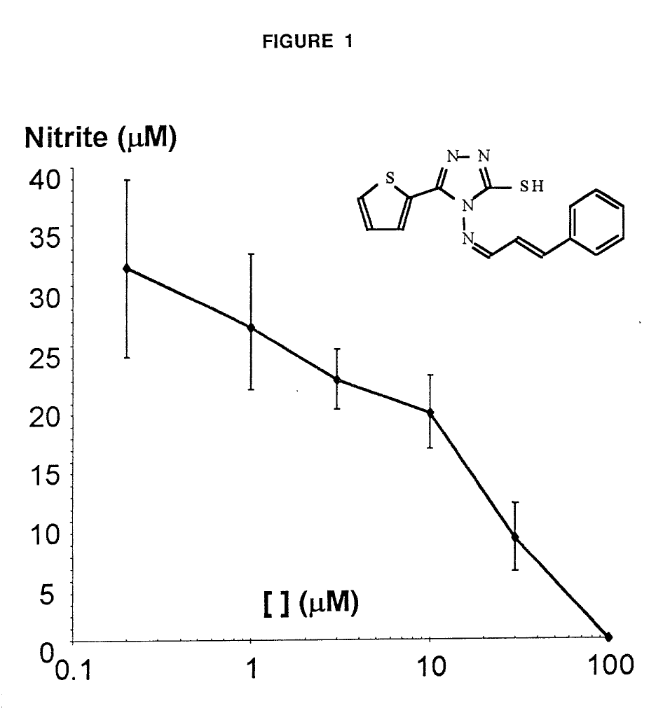

FIG. 1 depicts a typical response curve for inhibitors of nitric oxide synthase according to the present invention;

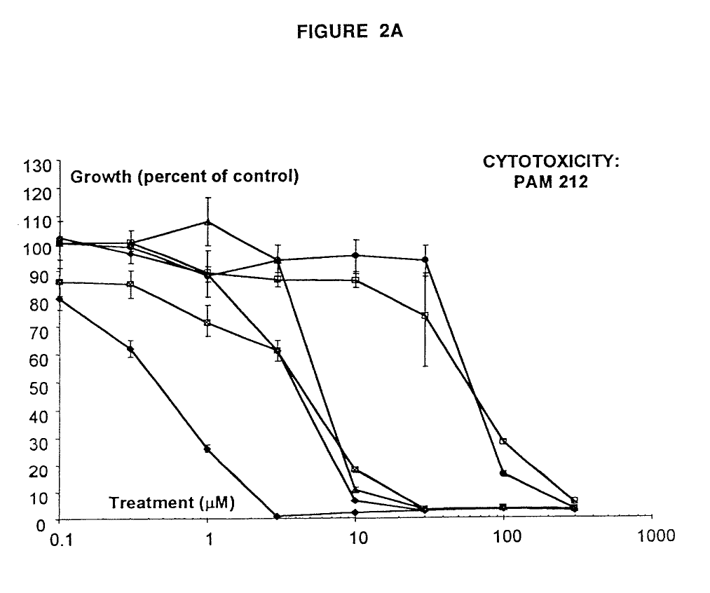

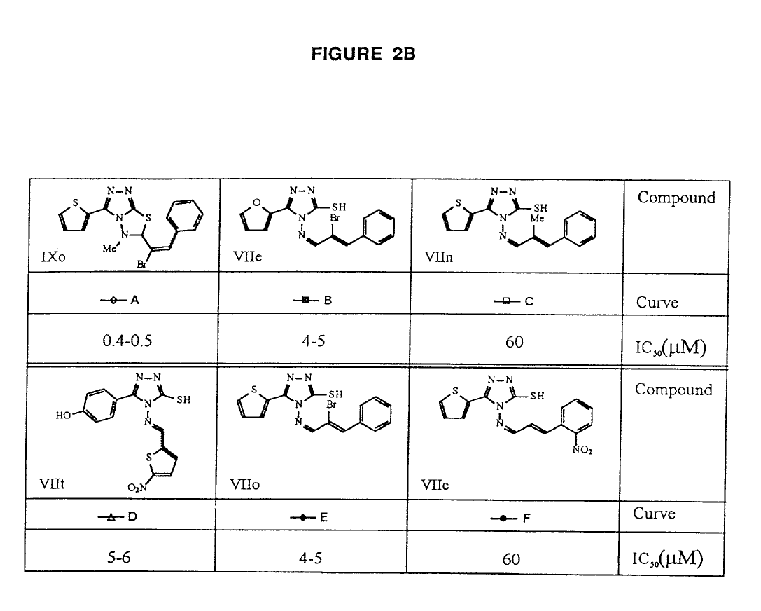

FIG. 2a depicts the results for inhibition of cellular growth using compounds according to the present invention, and

FIG. 2b depicts the compounds depicted in FIG. 2a along with their IC₅₀ concentrations in μM; and

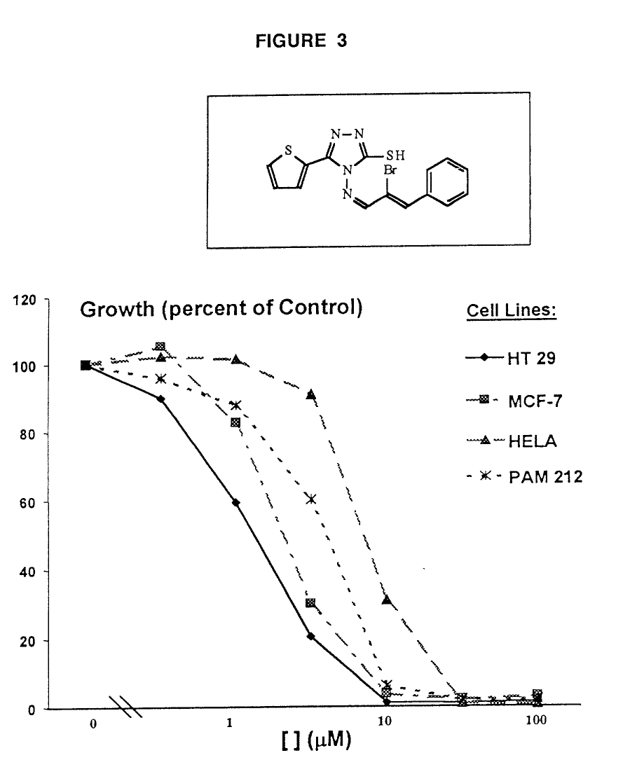

FIG. 3 depicts the decrease in growth, i.e., the inhibition of growth, brought about by one compound (compound VIIm) according to the present invention against various human cancerous cell lines.

In the following examples, all solvents, acids and other reagents were the highest purity grade available. NMR spectra, when provided, were obtained on a JEOL FX90Q spectrometer following the manufacturer's instructions. The synthesis of the starting 4-amino-3-mercapto-4H-5-R-1,2,4-triazoles were prepared using known techniques [see Journal of Heterocyclic Chemistry 13:925 (1976)].

### EXAMPLE 1

### Two-step Preparation of 3-R-8-aryl-1,2,4-triazolo[3,4-b]-1,3,4-thiadiazepines

### Thiadiazepines of General Formula IV

a) Preparation of the arylpropargyl aldehyde:

A suitably functionalized arylacetylene was condensed with triethyl orthoformate as catalyzed by zinc nitrate to yield the arylpropargyl aldehyde diethyl acetyl according to the method Houk and Sauer [see Journal of the American Chemical Society 80:4607 (1958)]. of 1739, 1459, 1377; ¹H NMR (DMSO): 1.34 (d, J=6.69 Hz, 3H), 2.89 (dg, J=16.0 Hz, J=8.21 Hz, J=6.14 Hz, 2H), 5.17 (m, 1H), 5.94 (s, 2H), 7.22 (t, J=4.93 Hz, J=3.78 Hz, 1H), 7.80 (d, J=4.93, 1H), 7.96 (d, 3.78, 1H).

Similarly, by this reaction compound VIb was obtained employing the corresponding triazole as defined by general formula III and phenyl crotonate. Compound VIc was obtained from the appropriate triazole defined by general formula III and methyl cinnamate.

The esters obtained as described above by the Michael reaction may be further functionalized by reduction and chlorination. For example, 4-amino-3-mercaptyl-(4-hydroxy-2-butyl)-5-(2-thienyl)-(4H)-1,2,4-triazole (compound VId) was prepared by the reduction of compound VIa following the following protocol:

The drop-wise addition of 4-amino-3-mercaptyl-(beta-methyl-butyrate)-5-(2-thienyl)-(4H)-1,2,4-triazole (compound VIa), 120 mg, (0.452 mmol) in 3 ml of THF was carried out into a solution of 17 mg of LiAlH₄ in 2 ml of THF under constant flow of nitrogen. The reaction mixture was stirred for 20 hours whereupon an additional 17 mg (0.452 mmol) of LiAlH₄were added to the mixture because it was not complete. After an additional 6 hours, another 34 mg (0.904 mmol) were added and the reaction was completed in 45 minutes. Subsequently, 1 ml of methanol, followed by 2 ml of water, was added to the reaction mixture to quench the excess LiAlH₄. The solvent was then removed under reduced pressure and an extraction was done with dichloromethane (3×, 20 ml) and water. The organic layers were combined, dried over magnesium sulfate, and the solvent was then removed under reduced pressure to give 61 mg of a white solid with IR (nujol) of 1592, 1449, 1051, 865; ¹H NMR (DMSO): 1.32 (d, J=6.70 Hz, 3H), 1.85 (m, 1H), 2.20 (m, 1H), 3.37 (m. 2H), 4.98 (m, 1H), 5.93 (s, 2H), 7.22 (dd, J=4.93 Hz, J=3.78 Hz, 1H), 7.80 (d, J=4.93, 1 H), 7.96 (d, J=3.78, 1H).

Crotonate, acrylate, and cinnamate esters, of a wide variety can be reduced to pendant side-chain bearing alcohols in this fashion without any detectable reduction of the hetercaromatic unsaturation. Yields of 35 to 55 can be expected.

Alcohols, for example such as compound VIb, may be chlorinated with triphenylphosphine and CCl₄ to the alkyl chlorides in conversions of 30 to 50%. Specifically, 4-amino-3-[(4-chloro-2-butyl)mercaptyl]-5-(2-thienyl)-(4H)-1,2,4-triazole (compound VIe) was prepared by the condensation of triphenylphosphine (154 mg,

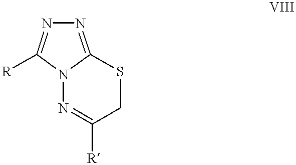

Third, the present invention describes a five-five fused ring system, specifically the 1,2,4-triazolo[3,4-b]-1,3,4-thiadazoles (general formula IX):

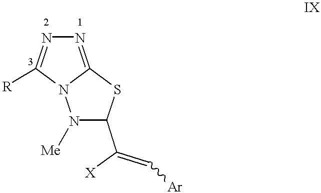

While a preparation of 3-R-8-aryl-1.2.4-triazole[3,4-b]-1,3,4-triadiazepines has previously been described [see Journal of Heterocyclic Chemistry 17:1087 (1980)], it is one aspect of the present invention to describe in the following examples a new and much more efficient route for the synthesis of these useful compounds.

It is another aspect of the present invention to describe a one-step synthesis of thiadiazepines of general formula IV in which (as depicted below) a ‘masked’ form of the requisite co-reactant arylporpargyl aldehyde (i.e., the arylacetylene diethyl acetal depicted below) is used and serves as an in situ precursor of the alkynyl aldehyde. This synthesis is a marked improvement over the earlier reported two-step synthesis for thiadiazepines [see OPPI, 123 (1980)]

Still another aspect of the present invention is to describe hereto unknown dihydro triazolothiadiazepines of general formula V.

Still another aspect of the present invention is to describe a selective reduction process of compounds of general formula IV to compounds of general formula V in which only one of the two possible double bonds is reduced.

| Compound | R | R′ | yield (%) | mp (° C.) |
|---|---|---|---|---|
| IVa | C₆H₅— | H | 27 | 240-250 |
| IVb | p-F—C₆H₄— | H | 54 | 217-219 |
| IVc | 2-thienyl | H | 26 | 164-166 |

### EXAMPLE 2

### One-step Preparation of 3-R-8-aryl-1,2,4-triazolo[3,4-b]-1,3,4-thiadiazepines

We have discovered that the arylacetylene diethyl acetals prepared by Houk and Sauer protocols could be used directly as synthetic equivalents of the arylpropargyl aldehydes. In this synthesis, a polar, anhydrous solvent (as, for example, tetramethylene-sulfolane, dioxane, monoglyme, or diglyme) is degassed by bubbling with nitrogen. The aminomercaptotriazole (according to general formula III), 0.050 moles, is added to the degassed solvent and the mixture refluxed with stirring under a nitrogen blanket for 15 minutes. These aminomercaptotriazoles were prepared as described in the literature [see Journal of Heterocyclic Chemistry 13:925 (1976), Journal of Organic Chemistry 45:2476 (1980), OPPI, 123 (1980), and Journal of Organic Chemistry 31:3528 (1966)]. The arylacetylene diethylacetyl is added (0.50 moles in the same solvent) and refluxed with a magnetic stirrer for 5 hours. Typical reaction ratios were 0.050-0.100 moles of the acetyl in 100-200 ml of solvent treated with an equimolar amount of aminomercaptotriazole in 50-100 ml of the same solvent.

In all these condensation procedures, some tarry material always accompanied the product formation. Crystals often precipitated from the reaction mixture while the stirring and refluxing were continued. Reaction conditions must be adjusted to minimize tarry by-products which often necessitated the removal of the reflux phase and only left simply stirring the acetal and aminomercaptotriazole at room temperature for 10 hours under a nitrogen blanket. Product isolation required evaporation of the solvent to half-volume, chilling in an ice/salt bath, and filtering the crude heterocycle. Crude product, often contaminated by tarry residue, could be purified by recrystallization from anhydrous ethanol or by dissolving the product in hot ethanol, adding decolorizing carbon, and filtering with vacuum through a moist pad of diatomaceous earth.

0.0589 mmol), triethylamine (83.4 μl, 0.589 mmol) and acetonitrile (2 ml) in a solution of 120 mg (0.455 mmol) of 4-amino-3-mercaptyl-(4-hydroxy-2-butyl)-5-(2-thienyl)-(4H)-1,2,4-triazole, 3 ml of acetonitrile, and 1 ml of carbon tetrachloride 20C. The reaction mixture was stirred for 1 hour at 0° C. after which the mixture was stirred for an additional 22 hours at room temperature. Since the reaction was not complete after 22 hours, the mixture was refluxed at 85-90° C. overnight. The product precipitated from the reaction mixture upon cooling and was isolated via suction, filtered, and washed with dichloromethane to give 45 mg of a tan solid. Additional properties of VIe are IR (nujol): 3486, 3412, 1634, 1459, 1380; ¹H NMR (D₂O): 1,46 (d, J=6.71 Hz, 3H), 2.25 (m, 1H), 2.44 (m, 1H), 3.35 (m, 2H), 4.51 (m, 1H), 7.15 (dd, J=4.95 Hz, J=3.97 Hz, 1H), 7.71 (d, J=4.95 Hz, 1H), 7.89 (d, J=3.78 Hz, 1H); ¹³C (proton coupled): 28.12 (q), 33.03 (t), 38.35 (t), 64.26 (d), 132.26 (s), 138.09 (d), 141.59 (d), 141.19 (d), 158.03 (s), 162.70 (s).

| Compound | R | R′ | yield (%) | mp (° C.) |
|---|---|---|---|---|
| VIa | 2-thienyl | β-Me-butyrate | 73 | 108-109 |
| VIb | 2-thienyl | β-phenyl-butyrate | 43 | 109.5-111 |
| VIc | 2-thienyl | β-phenylpropionate | 43 | 149.5-150 |
| | | methyl ester | | |
| VId | 2-thienyl | 4-hydroxy-2-butyl | 50 | 115-115.5 |
| VIe | 2-thienyl | 4-chloro-2-butyl | 34 | 196-197 |

### EXAMPLE 5

### Preparation of 4-(R′-imino)-3-mercapto-5-(R)-4H-1,2,4-triazole

### Triazoles of General Formula VII

Specifically, 4-imino-(cinnamyl)-3-mercapto-5-(2-thienyl)-4H-1,2,4-triazole (compound VIIa) was prepared by the condensation of 4-imino-3-mercapto-5-(2-thienyl)-4H-1,2,4-triazole, 100 mg (0.0504 mmol) with cinnamaldehyde, 123 mg (0.756 mmol) in 2 ml of absolute ethanol. The reaction was refluxed overnight, after which the product precipitated out of solution. The product was isolated via suction filtration and washed with cold ethanol to give 100 mg of product.

Similarly, by the same reaction a derivative of the 4-amino-5-(2-thienyl)triazole (compound VIIb, i.e., the o-methoxycinnamyl derivative) was obtained; and from the o-nitrocinnamyl derivative of the 4-amino compound was obtained Compound VIIc.

This reaction is, in fact, general for any 4-amino-3-mercapto-1,2,4-triazole, i.e., any compound defined by general formula III wherein R is methyl, cyclohexyl, phenyl, 4-fluorophenyl, 2-pyridyl, 3-pyridyl, 4-pyridyl, 2- or 3-furyl, or any other aryl, heterocyclic or alkyl moiety, in chemical condensation with any substituted cinnamaldehyde. Some specific examples (with R defined as shown and the substituted cinnamaldehyde component listed by name) are included in the following table:

| Com- | | | yield | |
|---|---|---|---|---|
| pound | R | R′ | (%) | mp (° C.) |
| VIIa | 2-thienyl | —CH═CH-Ph | 64 | 206-206.5 |
| VIIb | 2-thienyl | —CH═CH-(o-methoxy- | 75 | 227-228 |
| | | phenyl) | | |
| VIIc | 2-thienyl | —CH═CH-(o-nitro- | 83 | 214-215 |
| | | phenyl) | | |
| Com- | | Cinnamaldehyde | yield | |
| pound | R | compound | (%) | mp (° C.) |
| VIId | 2-furyl | cinnamaldehyde | 84 | 181-182 |
| VIIe | 2-furyl | α-bromocinnamaldehyde | 84 | 184-185 |
| VIIf | methyl | cinnamaldehyde | 59 | 193-194 |
| VIIg | methyl | α-bromocinnamaldehyde | 54 | 194-195 |
| VIIh | methyl | α-chlorocinnamaldehyde | 88 | 221.5-222.5 |
| VIIi | cyclohexyl | α-bromocinnamaldehyde | 97 | 199-200 |
| VIIj | Ph-CH₂CH₂— | α-bromocinnamaldehyde | 67 | 175-176 |
| VIIk | phenyl | α-bromocinnamaldehyde | 89 | 195-196 |
| VIIl | 4-F—C₆H₄— | cinnamaldehyde | 62 | 216-217 |
| VIIm | 4-F—C₆H₄— | α-bromocinnamaldehyde | 51 | 212-213 |
| VIIn | 2-thienyl | α-methylcinnamaldehyde | 45 | 200-201 |
| VIIo | 2-thienyl | α-bromocinnamaldehyde | 88 | 197-198 |
| VIIp | 2-thienyl | α-chlorocinnamaldehyde | 65 | 222-223 |

By way of the same experimental method shown above, employing a 1.0 to 1.5 ratio of any requisite member of the class of compounds defined by general formula III to any aromatic or heterocyclic aldehyde in sufficient anhydrous ethanol to achieve solubility, one can obtain 40 to 65% yields of purified members of chemical class VII (wherein R′ is an aromatic or heteroaromatic moiety). Addition of well-dried molecular sieves as water-absorbents increases the field and facilitates the reaction. Specifically, a mixture of 100 mg (0.52 mmoles) of general formula III (specifically wherein R was 4-hydroxyphenyl) and 123 (0.78 mmoles) of 5-nitro-2-thiophene carboxaldehyde in 10 ml of ethanol containing 40 mg of molecular sieves was refluxed for 72 hours, filtered hot, evaporated to about 5 ml, and chilled to obtain 89 mg of compound VIIt. Compound VIIt had the following additional properties: 1H NMR (CD₃COCD₃) ppm δ7.01 (d. J=8.9 Hz), 7.80-7.85 (m, 3Hβ/α′), 8.11 (d, J=3.8 Hz, Hβ′), 8.99 (s, —OH), 10.72 (s, —N═CH—).

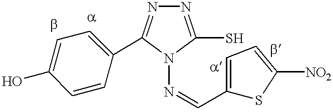

All imine members of class VII generated from aromatic and heterocyclic aldehydes in this fashion display a characteristic proton resonance for —N═CH— at 10.6±0.3 ppm. Additional examples of such non-cinnamyl imines (VIIq-r) are shown in the following table:

| | | Aromatic or | yield |
|---|---|---|---|
| Compound | R | heterocyclic aldehyde | (%) |
| VIIq | 3-pyridyl | 5-nitro-2-thiophene | 64 |
| | | carboxaldehyde | |
| VIIr | 3-pyridyl | 2,3-dihydroxybenzaldehyde | 48 |
| VIIs | 3-pyridyl | 4-chlorobenzaldehyde | 57 |
| VIIt | 4-OH—C₆H₄— | 5-nitro-2-thiophene | 49 |
| | | carboxaldehyde | |

Alkylation of the above indicated N-cinnamyl derivatives of those compounds defined by the structure of general formula VII possessing a ‘free’ thiol undergo reaction with alkylating species such as methyl iodide, dimethyl sulfate, ethyl iodide, and benzy tosylate to give a mixture of two heretofore unreported chemical families. In one of these families of compounds the alkylation occurs on the pendant imino nitrogen attached at N-4 of the triazole to produce a ring-closed family defined by general formula IX (i.e., cyclized N-alkyl analogs). In the minority alkylation pathway, attack occurs on the sulfur atom to produce S-alkyl analogs defined by general formula X. This reaction and separation are accomplished in the following fashion:

### EXAMPLE 6

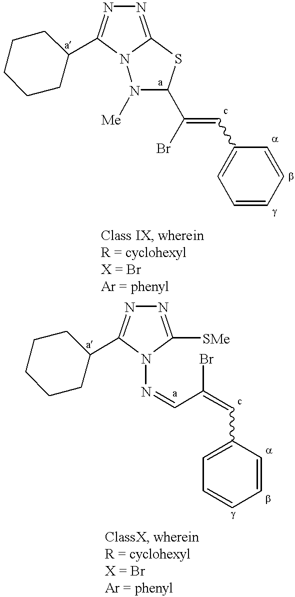

Specifically, 139 mg (0.036) of 4-imino-(α-bromocinnamyl)-3-mercapto-5-cyclohexyl-4H-1,2,4-triazole (compound VIIi, prepared in accordance with the process set forth in Example 5) was dissolved in 5 ml of dry acetone and reacted with 60 μl (0.096) methyl iodide in the presence of 133 mg (0.096) potassium carbonate for 3 hours at room temperature, the reaction flask being protected from light. The potassium carbonate was removed by filtration and the organic residue was filtered through a short column of flash silica gel. Elution with methylene chloride afforded 5 mg of a compound designated as Xi. Compound Xi had the following properties: ¹H NMR (CDl₃) δ: 1.23-1.46 (m, 3H); 1.5.-1.6 (m, 2H); 1.69-1.77 (m, 1H); 1.80-1.88 (m, 2H); 1.99-2.06 (m, 2H); 3.01 (t, J=11.6 Hz, J=3.3 Hz, Ha′); 3.75 (s, CH₃); 7.40-7.47 (m, 3Hβ/γ); 7.57 (s, Hc); 7.86-7.92 (m, 2Hα); 10.78 (s, Ha).

Evolution with 96% methylene chloride/4% methanol afforded 140.5 mg of a compound designated as IXi. Compound IXi had the following properties: ¹H NMR (CD₃COCD₃) δ: 1.26-1.44 (m, 3H); 1.60-1.72 (m, 3H); 1.78-1.87 (m, 2H); 1.95-2.02 (m, 2H); 2.66 (s, CH₃); 2.93 (t, J=13.2 Hz, J′+3.5 Hz, Ha′); 7.49-7.55 (m, 3Hβ/γ); 8.06 (s, Hc); 7.99-8.05 (m, 2Hα); 8.66 (s, Ha).

The ¹H NMR of both IXi and Xi show one characteristic singlet for Ha. In IXi, Ha undergoes an upfield shift of almost 2 ppm (from 10.46 ppm in the starting compound VIIi to 8.66 in Ixi) consistent with the change from an sp2 to an sp3 configuration for the carbon bearing that proton, whereas that same proton does not, as expected, undergo any significant shift in Xi (10.78 ppm). This correlation is general throughout the members of the class, and permits one to distinguish between N-alkylation/ring closure and S-alkylation.

Similarly, utilizing this reaction a derivative of the 4-amino-5-[2-(phenyl)ethyl]-triazole, i.e., compound IXa was obtained in a 96% yield, whereas the corresponding compound Xa was isolated in 4% yield from acetone.

Furthermore, from VIId was obtained IXd in 96% yield from acetone.

This reaction is general for any 4-(R′-imino)-3-mercapto-5-(R)-4H-1,2,4-triazole, i.e., any compound defined by general formula VII where R is methyl, cyclohexyl, phenyl, 4-fluorophenyl, 2-pyridyl, 3-pyridyl, 4-pyridyl, 2-furyl, 3-furyl, or any other aryl, heterocyclic, or alkyl function, and R′ is the attachment arising from a condensation of the N—NH₂ moiety with any substituted cinnamaldehyde.

Properties of other members of these two classes prepared in accordance with this example are contained in the following table:

| | | | | yield | |
|---|---|---|---|---|---|
| Compound | R | X | Ar | (%) | mp (° C.) |
| Xi | cyclohexyl | Br | phenyl | 3 | |
| Xj | Ph-CH₂CH₂— | Br | phenyl | 4 | |
| IXa | 2-thienyl | H | phenyl | 75 | 250-252 |
| IXd | 2-furyl | H | phenyl | 89 | 128-129.5 |
| Ixe (ex VIId) | 2-furyl | Br | phenyl | 96 | 169-170 |
| IXf | methyl | H | phenyl | 63 | 125-126 |
| IXg | methyl | Br | phenyl | 65 | 100-102 |
| IXh | methyl | Cl | phenyl | 92 | 78.5-80 |
| IXi | cyclohexyl | Br | phenyl | 97 | 83.5-84.5 |
| IXj | Ph-CH₂CH₂— | Br | phenyl | 96 | 115.5-116.5 |
| IXk | phenyl | Br | phenyl | 95 | 195-196 |
| IXo | 2-thienyl | Br | phenyl | 80 | 123-124 |
| IXp | 2-thienyl | Cl | phenyl | 62 | 130-131 |
| IXq | 2-thienyl | H | phenyl | 75 | |

### EXAMPLE 7

### Preparation of 3-(R′)-6-(R)-1,2,4-triazolo-[3,4-b]-1,3,4-thiadiazine

### Triazoles of General Formula VIII

Another class of triazole compounds according to the present invention having significant NOS inhibitory activity is the fused thiadiazine compounds of general formula VIII. Members of this class can be obtained in excellent yield by the condensation of the appropriate member of triazoles defined by general formula III with an alpha-haloketone. For example, 3-(2-thienyl)-8-phenyl-1,2,4-triazolo-[3,4-b]-thiadiazepine (general formula VIII wherein R is 2-thienyl and R′ is phenyl) was prepared by the dehydrative cyclization of 200 mg (1.009 mmol) 4-amino-3-mercapto-5-(2-thienyl)-4H-1,2,4-triazole with 156 mg (1.009 mmol) 2-chloroacetophenone in 10 ml of absolute ethanol at reflux for 2 hours. As the reaction mixture cooled to room temperature, the product precipitated. A cold, saturated solution of sodium acetate was added until the pH was 8, and the reaction mixture continued to be stirred overnight. The pale yellow solid was isolated via phosphorus pentoxide at 100° C. to give 287 mg (95% yield) of product with a melting point of 183-184° C. Additional properties of the product was IR (nujol): 1677, 1592, 1376, 691; ¹H NMR (CDCl₃): 4.03 (s, 2H), 7.17 (t, 1H), 7.51 (m, 4H), 7.96 (m, 3H).

Utilizing the procedure outlined above, the following members of the class defined by general formula VIII were obtained:

| Compound | R | R′ | yield (%) | mp (° C.) |
|---|---|---|---|---|
| VIIIa | 2-thienyl | phenyl | 95 | 183-184 |
| VIIIb | 2-thienyl | trifluoromethyl | 78 | 213-214 |
| VIIIc | methyl | trifluoromethyl | 69 | 85-86 |
| VIIId | methyl | phenyl | 76 | >230 |
| | | | | (decomp) |

### EXAMPLE8

### NOS Inhibition Assay

Compounds according to the present invention were assayed for pharmacological activity by examining their ability to inhibit nitric oxide production from PAM 212 keratinocytes stimulated to produce nitric oxide synthase with the cytokine gamma interferon as described in the literature [see Journal of Biological Chemistry 267:30 21277 (1992) and Biochemical Pharmacology 54:103 (1997)]. PAM 212 cells were maintained in growth medium consisting of Dulbecco's modified Eagles's medium (DMEM) supplemented with 10% fetal calf serum. For each assay, cells were inoculated into 24-well tissue culture plates (250,000 cells per well) in growth medium. After 24 hours, the medium was changed to phenol red and serum-free DMEM containing 100 U/ml of gamma interferon. After 72 hours, nitric oxide production by the cells was quantified spectrophotometrically by measuring the accumulation of nitrite in the culture medium using the Greiss reagent. An aliquot of the culture medium was mixed with equal volumes of 1.0% sulfanilamide and 0.1% N-1-naphthylethylene diamine in 50% phosphoric acid. After 15 minutes at room temperature, the absorbance of the resulting chromophore was measured at 540 nm using a microplate reader and the results compared to standard solutions of sodium nitrite.

A typical response curve for inhibitors of nitric oxide synthase (specifically for compound VIIa) is shown at FIG. 1.

Members of all triazole and fused-ring triazole families according to the present invention (i.e., members belonging to families defined by general formulae III, IV, V, VI, VII, VIII, IX, and X) displayed NOS-inhibiting activity in this assay. Typical values as IC₅₀'s obtained from the testing and graphical analysis described above were:

| Compound | R | R′ | μM |
|---|---|---|---|
| III | 4-F—C₆H₄— | | 98 |
| III | 2-thienyl | | 77 |
| IV | 4-F—C₆H₄— | H | 21 |
| IV | 2-thienyl | H | 48 |
| IV | benzyl | H | 52 |
| IV | 2-Br—C₆H₄— | H | 87 |
| IV | 2-thienyl | Br | >100 |
| V | C₆H₅— | H | 110 |
| VI | methyl | —CH₂—CH₂—COOMe | 170 |
| VI | 2-thienyl | —CH(CH₃)CH₂CH₂Cl | >100 |
| VI | 2-thienyl | —CH(CH₃)CH₂CO₂(C₆H₅) | >100 |
| VII | 2-thienyl | —CH═CH-Ph | 35 |
| VII | 2-thienyl | —CH═CH-2-methoxyphenyl | >100 |
| VII | 2-thienyl | —CH═CH-2-nitrophenyl | 12 |
| VII | 3-pyridyl | 2,3-dihydroxyphenyl | 29 |
| VIII | methyl | trifluoromethyl | >100 |
| VIII | 4-F—C₆H₄— | trifluoromethyl | 30 |
| VIII | 4-F—C₆H₄— | phenyl | >100 |
| IX | 2-thienyl | H (Ar = phenyl) | 15 |
| IX | methyl | H (Ar = phenyl) | 17 |
| IX | 2-furyl | H (Ar = phenyl) | 14 |
| X | cyclohexyl | Br (Ar = phenyl) | 146 |

Members of the triazole families according to the present invention also have the ability when screened in an in vitro assay of inhibiting the proliferation of tumor cells grown in culture, an excellent indication of potential in vivo activity. Representative members of the triazole families according to the present invention were tested for biological activity in this assay and found to be potent inhibitors of cell growth inhuman colon carcinoma (HT29 cells), in breast cancer (MCF-7 cells), in cervical cancer (HeLa cells), and in skin cancer (PAM 212 cells). Clearly, therefore, these findings directly demonstrate that the compounds according to the present invention are potential therapeutics for human proliferative diseases. A description of this assay on PAM 212 cells, as an example, follows:

### EXAMPLE 9

### Antiproliferative Assay

The ability of any one of the compounds according to the present invention to inhibit cell growth is recognized by the scientific community to be directly related to its therapeutic potential as an anticancer agent. This type of growth assay can be used with mammalian cancer cells as well as with pathogenic and non-pathogenic microbes, including, but not limited to, yeasts and bacteria.

To assay these compounds for anticancer activity, tumor cells (PAM 212) grown in vitro in monolayer culture flasks were used. Cells were inoculated into 6-well culture dishes (3.5 cm diameter wells, 25,000 cells per well) in 2 ml of growth medium consisting of Dulbecco's modified Eagle's medium supplemented with 10% calf serum. After 24 hours at 37° C. in a humidified incubator with an atmosphere containing 5% carbon dioxide, the growth medium was drained from the cells and replaced with 2 ml of growth medium containing either control vehicle or increasing concentrations of the candidate anticancer agents. Triplicate wells on the plates were used to measure control growth and growth of the cells in the presence of each concentration of anticancer agent. The cells were then returned to the incubator. After the cells had grown for 4 to 5 days, the medium was drained from the culture dishes and the cells washed with phosphate buffered saline.

The cells from each well on the culture dishes were removed by trypsin treatment and counted with a Coulter counter. After a period of time, the number of cells in each well was determined. The data can be presented as a curve showing the inhibition of tumor cell growth with increasing concentrations of the test compound. The concentration inhibiting cell growth by 50% (IC₅₀) was determined from the curve. Each of the compounds according to the present invention was a potent inhibitor of cell growth with the IC₅₀ values being typically in the micromolar concentration range.

When tested in accordance with this protocol, members of triazole families VII and IX for example, displayed cellular growth inhibition properties when tested against PAM 212 cells according to the following table some results of which may also be found in FIGS. 2A and 2B:

| Compound | R | R′ | X | Ar | μM |
|---|---|---|---|---|---|
| VII | 2-thienyl | —C(CH3)═CH-Ph | | | 60 |
| VII | 2-thienyl | —CBr═CH-Ph | | | 5 |
| VII | 2-thienyl | —CH═CH-2- | | | 60 |
| | | nitrophenyl | | | |
| VII | 2-furyl | —CBr═CH-Ph | | | 4.5 |
| VII | methyl | —CBr═CH-Ph | | | 6 |
| IX | 2-thienyl | | H | phenyl | 44 |
| IX | 2-thienyl | | Br | phenyl | 0.4-0.5 |
| IX | 2-thienyl | | Cl | phenyl | 4 |
| IX | 2-furyl | | H | phenyl | 45 |
| IX | methyl | | H | phenyl | >100 |

When member VIIo, i.e., general structural formula VII wherein R is 2-thienyl and R′ is —CBr═CH—Ph, was tested as above but with different cell lines, the following results were obtained (see FIG. 3):

| Cell Line | IC₅₀ (μM) |
|---|---|
| HT 29 | 1.3 |
| MCF-7 | 2.5 |
| HELA | 8 |
| PAM 212 | 5 |

As noted above, aspects of the present invention involve a pharmacologically acceptable composition for inhibiting nitric oxide synthase in a mammal and inhibition of cancer cell growth. This composition comprises members of triazole families defined by general formulae III to X in amounts sufficient to be pharmaceutically active for the intended purpose, either in pure form or formulated together with one or more conventionally recognized pharmaceutically acceptable carriers, diluents, fillers, buffering agents, flavorants, binders, lubricants, thickening agents, polyethylene glycol or any other conventional materials used in the manufacture of pharmaceutical preparations.

Pharmaceutical formulations of the triazole members according to the present invention may include those suitable for oral, rectal, nasal, topical (including buccal and sub-lingual), ocular, vaginal, parenteral (including intramuscular, subcutaneous, and intravenous) administration, or for administration by inhalation or insufflation. The formulations may, where appropriate, be conveniently presented in discrete dosage units and may be prepared by any of the methods well known in the pharmaceutical art.

In short, the compounds useful in this invention may be administered transdermally, and by the term “transdermal” is meant any method by which the members of the triazole families according to the present invention are introduced across an epidermal layer of cells. For example, transdermal as used in this disclosure encompasses the administration of the compound by topical methods; by intravenous, intramuscular or subcutaneous injection; by solution for use as ocular drops, nasal sprays or tracheal sprays; by the oral route of administration such as by pills, troches, etc.; and by suppositories for vaginal or anal routes of administration. The compound will be formulated in suitable compositions determined by the intended means of administration, according to methods and procedures well-known to those skilled in the art. For example, the compounds suitable for use in this invention may be formulated or compounded into pharmaceutical compositions comprising at least one compound of the present invention (the compositions according to the present invention may comprise one compound or admixtures of compounds according to the present invention) in admixture with a solid or liquid pharmaceutical excipeint such as a diluent or carrier for enteral or parenteral administration. As injection medium, water containing the usual pharmaceutical additives for injection solutions, such as stabilizing agents, solubiliizing agents, and buffers is preferred. Among additives of this type are, for example, tartrate and citrate buffers, ethanol, complex forming agents such as ethylenediamine-tetraacetic acid, and high molecular weight polymers such as liquid polyethylene oxide for viscosity regulation. Solid carrier materials include, for example, starch, lactose, mannitol, methyl cellulose, talc, highly dispersed silicic acid, high molecular weight fatty acids such as stearic acid, gelatin, agar-agar, calcium phosphate, magnesium stearate, animal and vegetable fats, and high molecular weight polymers such as polyethylene glycols. Compositions suitable for oral administration can, if desired, contain flavoring and/or sweetening agents. For topical administration, the compounds may be preferably used with various conventional bases for topical preparations such as creams, ointments, gels, lotions, or sprays, depending upon the desired mode of delivery of the ingredients to an individual. In manufacturing these preparations, the composition may also be mixed with conventional inert excipients such as thickening agents, emollients, surfactants, pigments, perfumes, preservatives, fillers, and emulsifiers, all of which are well known and conventionally used in the formulation of transdermal or other preparations. Typically, these nonactive ingredients will make up the greater part of the final preparation. Preferably, the compositions are manufactured to allow for slow-release or timed-release delivery.

The actual amount of administered compound according to the present invention may vary between fairly wide ranges depending upon the mode of administration, the excipients used, the age and weight of the patient, and the severity of the condition being treated. While the precise amount administered to a mammalian patient is well within the discretion of the attending physician, such unit dosage amounts administered will normal be from 1 to 250 mg/kg weight of the mammalian patient/day. Such amounts are well within the skill of the pharmaceutical scientist to prepare and the physician to administer.

Thus while we have illustrated and described the preferred embodiment of our invention, it is to be understood that this invention is capable of variation and modification, and we therefore do not wish to be limited to the precise terms set forth, but desire to avail ourselves of such changes, modifications and alterations which may be made for adapting the invention to various usages and conditions. Accordingly, such changes, modifications and alterations are properly intended to be within the full range of equivalents, and therefore within the purview of the following claims.

Having thus described our invention and the manner and a process of making and using it in such full, clear, concise and exact terms so as to enable any person skilled in the art to which it pertains, or with which it is most nearly connected, to make and use the same; we claim:

1. A triazole compound having a general structural formula of: 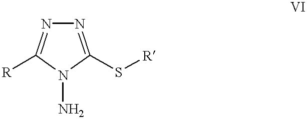
wherein R and R′, which may be the same or different, are alkyl, aryl, hydrogen, haloalkyl, or heterocyclic moieties, with the proviso that R′ is not hydrogen.

2. A triazole according to claim 1 wherein R′ is a substituted β-carboalkoxy ethyl residue.

3. A triazole compound having a general structural formula selected from the group: 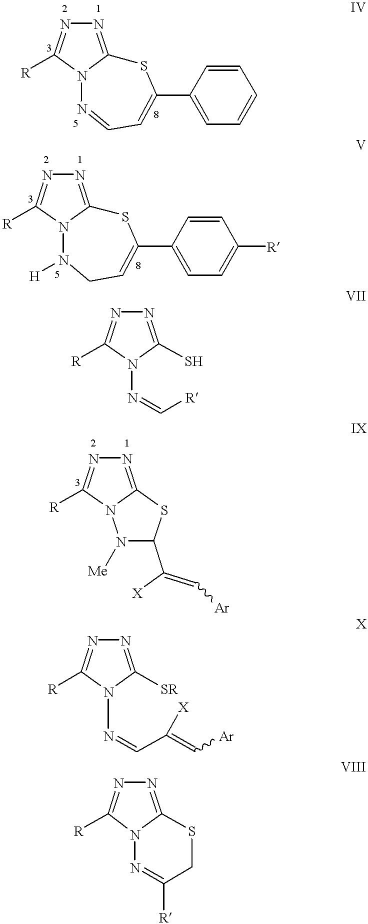
wherein R and R′, which may be the same or different, are alkyl, aryl, hydrogen, haloalkyl, or heterocyclic moieties; X is a halogen; and Ar is an aryl moiety.

4. A method for the synthesis of 3-R-8-aryl-1,2,4-triazolo[3,4-b]-1,3,4-thiadiazepines which comprises:
degassing a polar, anhydrous solvent;
adding an aminomercaptotriazole to the degassed solvent;
adding an arylacetylene diethyl acetal to the resulting reaction mixture;
allowing the mixture to react; and
removing the thiadiazepine product from the reacted mixture.

5. A method for inhibiting nitric oxide synthase in a cell which comprises contacting said cell with at least one compound having the general structural formula of the group consisting of: 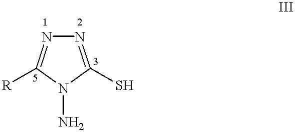
and as defined by the structual formulae of claims 1 and 3 in an amount sufficient to bring about said inhibition of nitric oxide synthase in said cell.
